(19) United States
(12) Patent Application Publication
(10) Pub. No.: US 2005/0176672 A1
(43) Pub. Date: Aug. 11, 2005

(54) Use of cationic lipids to generate anti-tumor immunity

(75) Inventors: Ronald K. Scheule, Hopkinton, MA (US); Nelson S. Yew, West Upton, MA (US); Lee Mizzen, Victoria (CA); Salam Abdul Kadhim, Kirkland (CA)

Correspondence Address: GENZYME CORPORATION;LEGAL DEPARTMENT, 15 PLEASANT ST CONNECTOR, FRAMINGHAM, MA, 01701-9322, US

(73) Assignees: Genzyme Corporation; Stressgen Biotechnologies

(21) Appl. No.: 11/100,038

(22) Filed: Apr. 6, 2005

Related U.S. Application Data
(63) Continuation of application No. 09/890,712, filed on Nov. 9, 2001, now abandoned.
(60) Provisional application No. 60/118,802, filed on Feb. 5, 1999.

(51) Int. Cl. A61K 48/00

(52) U.S. Cl. 514/44

(57) ABSTRACT

A method of generating an anti-tumor immune response using a cationic molecule:biologically active molecule complex is provided. In one embodiment, the anti-tumor immune response is a protective, memory-based response. The complex may be administered alone, as the active ingredient in a formulation, or as an adjuvant. The invention also provides for methods of generating an immunostimulatory response against the tumor cell present during treatment by exposing a cationic molecule:biologically active molecule complex to a mammalian cell or a foreign tumor cell.

This application is a U.S. National Phase Application based on PCT/US00/02943, filed Feb. 4, 2000, in the English language and claims the benefit of U.S. Provisional Application No. 60/118,802, filed Feb. 5, 1999, the content of both of which is incorporated herein by reference.

The present invention relates to a novel method of suppressing tumor growth and generating protective immunity against tumor recurrence. The present invention also relates to methods and compositions for modulating inflammatory responses in mammals and generating specific immunostimulatory responses.

Lipid mediated gene delivery has become one of the most widely researched areas of gene therapy. Cationic molecules, herein defined as cationic lipids, cationic polymers, and cationic amphiphiles have demonstrated particular promise for efficient intracellular delivery of biologically active molecules. Cationic molecules have polar groups that are capable of being positively charged at or around physiological pH. This property is understood in the art to be important in defining how the molecule interacts with many types of biologically active molecules including, for example, negatively charge polynucleotides such as DNA.

Examples of cationic lipid compounds that are stated to be useful in the intracellular delivery of biologically active molecules can be found throughout the literature along with discussions of properties of cationic lipids that are understood in the art as making them suitable for such applications. The disclosures of several of the examples found in the literature are specifically incorporated by reference herein. (U.S. Pat. No. 5,283,185 to Epand et al.; U.S. Pat. No. 5,264,618 to Felgner et al.; U.S. Pat. No. 5,334,761 to Gebeyehu et al.; and Lee, E. R. et al., Hum. Gene Ther. 7: 1701-1717 (1996)).

Another class of cationic lipids with enhanced activity is described, for example, in U.S. Pat. No. 5,747,471 to Siegel et al., U.S. Pat. No. 5,650,096 to Harris et al., and PCT publication WO 98/02191 published Jan. 22, 1998, the disclosures of which are specifically incorporated by reference herein. These patents also disclose formulations, characteristics and properties of cationic lipids of relevance to the practice of the present invention.

Additionally, several issued U.S. Patents, the disclosures of which are specifically incorporated by reference herein, have described the utility of cationic lipids to deliver polynucleotides to mammalian cells. (U.S. Pat. No. 5,676,954 to Brigham et al. and U.S. Pat. No. 5,703,055 to Felgner et al.)

However, an inflammatory response associated with lipid gene delivery has been recognized. For example, cationic lipid-mediated gene transfer to the lung induces dose dependent pulmonary inflammation characterized by an influx of leukocytes (predominantly neutrophils) and elevated levels of inflammatory cytokines such as interleukin-6 (IL-6), tumor necrosis factor a (TNF-a), and interferon-g (TNF-g) in the bronchoalveolar lavage fluid. Histopathological analysis of lung sections treated with the individual components of cationic lipid:DNA complexes suggests that the cationic lipid was a mediator of the observed inflammation.

Additionally, results of clinical studies where CF subjects were subjected to either aerosolized liposomes alone or cationic lipid:DNA complexes indicated that bacterial derived plasmid DNA may also be inflammatory. Each of the cationic lipid:pDNA-treated patients exhibited mild flu-like symptoms (including fever, myalgia, and a reduction in FEV of approximately 15%) over a period of approximately 24 hours. These symptoms were not observed in patients treated with the liposome control. One possible explanation for this response is related to the presence of unmethylated CpG dinucleotide sequences in bacterially-derived pDNA. See Krieg et al., Nature 374: 546-549 (1995); Klinman et al., Proc. Natl. Acad. Sci. USA 83: 2879-2883 (1996); Sato et al., Science 273: 352-354 (1996).

Short regions of genome consisting of unmethylated CpG dinucleotides are known as CpG islands or CpG motifs. Unmethylated CpG dinucleotides are present at a much higher frequency in bacterially-derived plasmid DNA compared to vertebrate DNA and are sometimes characterized as a subtle structural difference between bacterial and vertebrate DNA. For example, compared to DNA of eukaryotic origin, bacterial genomic DNA may contain a 20 fold higher frequency of the dinucleotide sequence CpG. Additionally, unlike eukaryotic DNA where 80% of the cytosines are methylated, those derived from prokaryotic origin are relatively unmethylated. These differences purportedly allow the vertebrate immune system to recognize and respond to DNA of bacterial origin. In this regard, administration of genomic bacterial DNA into an eukaryotic host has been shown to be capable of eliciting a potent immunostimulatory response. See Krieg et al., Trends Microbiol. 4: 73-76 (1995); Ballas et al., J. Immunol. 157: 1840-1845 (1996); Sparwasser et al., Eur. J. Immunol. 27: 1671-1679 (1997).

Consequently, CpG motifs of bacterial and synthetic dinucleotides have found many uses. The presence of CpG motifs is thought to activate certain immune cells, including B cells, monocytes, dendritic cells, macrophages, and natural killer cells. CpG motifs can also be used to activate protective immune responses against infection, enhance vaccines, activate the immune system against cancer cells, and convert allergic reactions into harmless responses. See Wooldridge et al., Blood 89: 2994-2998 (1997).

Systematic analysis of CpG motifs has indicated that those sequences harboring the CpG motif 5′-RRCOYY-3′ were particularly potent at inducing these responses. It was demonstrated that this effect was a consequence of the methylation status of the CpG dinucleotide sequences by experiments showing that administration of either bacterial genomic DNA or synthetic oligonucleotides bearing the RRCOYY sequence that had been pre-methylated with CpG methylase were significantly less immunostimulatory.

Since plasmid DNA used in gene transfer studies is usually isolated from bacterial sources, and because it also harbors bacterial sequences for propagation in the host, it contains a higher frequency of unmethylated CpG sequences. Subsequently, the presence of CpG motifs has been detrimental to the effective introduction of many types of biologically active molecules in gene therapy. For example, the generation of elevated levels of cytokines due to CpG motifs in the BALF has consequences for expression of the therapeutic protein. Several viral promoters, such as the CMV promoter commonly used in gene delivery vectors, are subject to suppression by such cytokines. Furthermore, any additional inflammation or reduction in lung function in patients that already exhibit chronically inflamed, compromised airways represents an increased safety risk.

The presence of CpG motifs on pDNA has also been shown to be capable of stimulating a robust T-helper 1 type response in either transfected monocytes or injected BALB/c mice. Of particular concern for delivery of genes to the lung was the demonstration that bacterial genomic DNA or oligonucleotides containing immunostimulatory CpG motifs are capable of eliciting an acute inflammatory response in airways and in particular caused inflammation in the lower respiratory tract, increasing both cell numbers and elevated levels of the cytokines TNF-α, IL-6 and macrophage inflammatory protein (MIP-2). See Schwartz et al., J. Clin. Invest. 100: 68-73 (1997).

Activation of a similar cytokine profile by CpG dinucleotides have also been reported in murine dendritic cells (Sparwasser et al., Em. J. Immunol. 28: 2045-2054 (1998)), macrophages (Lipford et al., Em. J. Immunol. 27: 2340-2344 (1997)), monocytes (Sato et al., Science 273: 352-354 (1995)), and NK cells (Cowdery et al., J. Immunol. 156: 4370-4575 (1996)). A recent study also reported that complexes formed between the cationic lipid DOTMA (N-[1-(2-3-dioleyloxy)propyl]-N,N,N-trimethylammonium chloride) and pDNA enhanced cytokine and cellular levels in the BALF of treated animals. See Friemark et al., J. Immunol 160: 4580-4586 (1998).

### SUMMARY OF THE INVENTION

The present invention provides for a method of generating an anti-cancer effect in a mammal by administering an effective amount of composition comprising a cationic molecule and a biologically active molecule for the purpose of stimulating an anti-tumor cell response. In a preferred embodiment, the composition comprises a cationic lipid:biologically active molecule complex. In a further preferred embodiment, the biologically active molecule is an immunologically active nucleic acid sequence with or without an expressible cDNA insert.

In a further preferred embodiment, the anti-cancer effect may be an anti-tumor cell response including an apoptotic response, an anti-angiogenic response, or an immune response including an inflammatory response, a humoral response, a cellular response, a Th1-type response, or a Th2-type response.

A subject of the invention is also a method of modulating an immune response in a mammal by administering an effective amount of a composition comprising a cationic molecule and a biologically active molecule, for the purpose of modulating the immune response. The composition may comprise a cationic lipid:biologically active molecule complex and the biologically active molecule may be an immunologically active nucleic acid sequence with or without an expressible cDNA insert. In a preferred embodiment the immune response may be an inflammatory response, a humoral response, a cellular response, a Th1-type response, or a Th2-type response.

Also within the practice of the invention is a method of generating an anti-tumor response in a mammal by contacting a tumor cell with an effective amount of composition comprising a cationic molecule and a biologically active molecule, for the purpose of generating the anti-tumor response. In a preferred embodiment, the anti-tumor response is a protective anti-tumor immune response that may provide long term protective immune memory. The composition may comprise a cationic lipid:biologically active molecule complex and in a further preferred embodiment the anti-tumor response is a systemic response. Another subject of the invention is the generation of a systemic immune response by administering an effective amount of a composition comprising a cationic lipid and a biologically active molecule to an environment containing a tumor cell in a mammal.

The practice of the invention also provides for a composition effective for generating an immune response against the tumor cell present during treatment. The composition comprises a cationic molecule and a biologically active molecule. Preferably the composition of the invention comprises a cationic lipid:biologically active molecule complex. The invention provides for the delivery of these compositions to a mammal to stimulate an inflammatory response and/or immune response. In a preferred embodiment, the invention provides for a method of stimulating an inflammatory and/or immune response by delivering a composition comprising a an immunologically active nucleic acid sequence which may be a bacterial plasmid.

The invention further provides for delivery of a cationic molecule:biologically active molecule complex to a compartment containing a tumor cell, or to a tumor cell itself by any methods known in the art to deliver a biologically active molecule.

In a further embodiment, the invention provides for compositions which are effective for stimulating an inflammatory response or an immune response against the tumor cell present during treatment using a biologically active molecule that comprises an immunologically active nucleic acid sequence, which mayor may not contain an expressible cDNA insert. Thus, the methods described above do not require the expression of a transgene.

In another aspect, the invention provides for pharmaceutical compositions comprising a cationic molecule:biologically active molecule complex which stimulates an inflammatory, immune, or anti-tumor response. The compositions may be an active ingredient in a pharmaceutical composition that includes carriers, fillers, extenders, dispersants, creams, gels, solutions and other excipients that are common in the pharmaceutical formulatory arts. The pharmaceutical compositions may be delivered to a tumor cell or they may be delivered to an environment containing a tumor cell in order to stimulate an immune response against the tumor cell present during treatment.

In a further embodiment, the invention provides for the use of a cationic molecule:biologically active molecule complex as an adjuvant that may be used in combination with another drug or treatment to increase or aid its effect. Examples of drugs or other treatments that may be utilized in combination with a cationic lipid:biologically active molecule complex include but are not limited to known tumor antigens, surgery, cytokines or any treatment that does substantially compromise an immune response.

The invention provides for a method of administering the compositions by any methods that have been employed in the art to effectuate delivery of biologically active molecules to the cells of mammals including but not limited to administration of an aerosolized solution, intravenous injection, or oral, parenteral, intra-peritoneal, intra-nasal, topical, or transmucosal administration.

The invention also provides for a pharmaceutical composition that comprises one or more lipids or other carriers that have been employed in the art to effectuate delivery of biologically active molecules to the cells of mammals, and one or more biologically active molecules, wherein said compositions facilitate intracellular delivery to the cells, tissues or organs of patients of an effective amount of the cationic molecule:biologically active molecule complex. The pharmaceutical compositions of the invention may be formulated to contain one or more additional physiologically acceptable substances including components that: stabilize the compositions for storage; target specific tissues, cells, membranes, or organs in the subject; and/or contribute to the successful delivery of the cationic molecule:biologically active molecule complex.

For pharmaceutical use, a cationic lipid:biologically active molecule complex of the invention may be formulated with one or more additional cationic lipids including those known in the art, or with neutral co-lipids such as dioleoylphosphatidyl-ethanolamine, (“DOPE”), to facilitate delivery to cells the cationic lipid:biologically active molecule complex.

Additional features and advantages of the invention will be set forth in the description which follows, and in part will be apparent from the description, or may be learned by the practice of the invention. The objectives and other advantages of the invention will be realized and attained by the compounds and methods particularly pointed out in the written description and claims hereof as well as the appended drawings.

### BRIEF DESCRIPTION OF THE DRAWINGS

FIG. 1. Cytokine analysis of mouse BALF after instillation of GL-67 complexed with methylated or unmethylated pCF1-CAT. Groups of three BALB/c mice were instilled intra-nasally with 100 μl of GL-67:(m)pCF1-CAT, GL-67:pCF1-CAT, GL-67 alone, (m)pCF1CAT, pCF1-CAT, or vehicle (naive). BALF was collected 24 h after instillation and ELISA assays were used to measure the levels of various cytokines. (m)pCF1-CAT refers to pCF1-CAT that had been methylated by Sss I methylase.

FIG. 2. Total cell counts (FIG. 2A) and proportion of neutrophils (FIG. 2B) in BALF after administration of cationic lipid:pDNA complexes. Groups of three BALB/c mice were instilled intranasally with 100 μl of GL-67:(m)pCF1-CAT, GL-67:pCF1-CAT, GL-67 alone, (m)pCF1-CAT, pCF1-CAT, or vehicle. BALF was collected 24 h post-instillation and total cells and the different cell types were counted. (m)pCF1-CAT refers to pCH-CAT that had been methylated by Sss I methylase while PMN, refers to polymorphonuclear leukocytes.

FIG. 3. Cytokine analysis of mouse BALF after instillation of GL-67 complexed with mixtures of methylated and unmethylated pCF1-CAT. Sss 1-methylated pCF1-CAT was mixed with unmethylated pCF1-CAT at ratios of 0:3, 1:2, 2:1, or 3:0 [(m)pCF1-CAT:pCF1-CAT], then complexed with GL-67 to final concentration of 0.3:1.8 MM (GL-67:pDNA). Groups of three BALB/c mice were instilled intranasally with 100 μl of GL-67:pDNA complexes and BALF was collected 24 h after instillation for cytokine assays. Naive animals were treated with vehicle. (m) refers to methylated pCF1-CAT while (m) refers to non-methylated pCF1-CAT.

FIG. 4. Histopathological analysis of BALB/c mouse lung sections following administration of GL-67 complexed with methylated or unmethylated pCF1-CAT. BALB/c mice were instilled intranasally with μl of GL-67:(m)pCF1-CAT, GL-67:pCF1-CAT, GL-67 alone, (m)pCF1-CAT, pCF1-CAT, or vehicle. Mice were sacrificed two days post instillation and the lungs were processed for histological examination in a blinded manner. Lung inflammation was graded on a scale of 0 to 4, with 0 indicating no change, 1 a minimal change, 2 a mild change, 3 a moderate change, and 4 indicating a severe change nom a normal lung. (m)pCF1-CAT refers to pCF1-CAT that had been methylated by Sss I methylase.

FIG. 5. CpG motifs present in pCF1-CAT. The motifs having the sequence 5′-RRCGYY-3′ are as shown. Numbers in parentheses indicate the nucleotide position of the cytosine residue. The figure uses the following abbreviations: Kan R, the gene for kanamycin; CMV Promoter, cytomegalovirus promoter; CAT, cDNA for cloramphenicol aceyltransferase; BGH PolyA, polyadenylation sequence from bovine growth hormone.

FIG. 6. Relative levels of CAT expression following methylation or mutagenesis of pCF1-CAT Groups of three BALB/c mice were instilled intranasally with 100 μl of GL67:pCF1-CAT, GL-67:(m)pCF1-CAT, GL-67:pCFA-299-CAT, or GL-67:pCFA-299-10M-CAT. pCFA-299-CAT harbors a partial deletion of the CMV promoter and pCFA-299-10M-CAT, an additional 10 mutations at CpG sites harboring the sequence motif RRCGYY. (m)pCF1-CAT refers to pCF1-CAT that had been methylated by Sss I methylase. Lungs were harvested for CAT analysis at day 2 post-instillation.

FIG. 7. Cytokine analysis of mouse BALF after instillation of GL-67 complexed with pCF1-CAT and modified forms of pCF1-CAT containing reduced numbers of CpG motifs. Groups of three BALB/c mice were instilled intranasally with 100 μl of GL-67:pCF1-CAT, GL-67:(m)pCF1-CAT, GL-67:pCFA-299-CAT, or GL-67:pCFA-299-10M-CAT. BALF was collected 24 h after instillation and ELISA assays for TNF-α, IFN-γ, IL-6, and IL-12 were performed. (m)pCF1-CAT refers to pCF1-CAT that had been methylated by Sss I methylase. pCFA-299-CAT harbors a partial deletion of the CMV promoter and pCFA-299-10M-CAT, an additional 10 mutations at CpG sites harboring the sequence motif RRCGYY.

### DETAILED DESCRIPTION OF THE INVENTION

In the present invention, a cationic molecule:biologically active molecule complex is used to generate an anti-cancer or anti-tumor effect and in a preferred embodiment the antitumor effect is generated by stimulating or modulating an immune or inflammatory response in a mammal. The complex may be administered alone, as the active ingredient in a formulation, as an adjuvant, or as part of a composition with another carrier such as a lipid, including cationic lipids, viral vectors, including adenoviruses, and other methods that have been employed in the art to effectuate delivery of biologically active molecules to the cells of mammals.

In one subject of the invention, the methods of stimulating and/or modulating an immune response by delivering a cationic molecule:biologically active molecule complex to a cell is for the purpose of generating a systemic immune response. The invention provides for the delivery of any cationic molecule:biologically active molecule complex to a mammalian cell to stimulate an inflammatory response and/or an immune response. The invention also provides for methods of generating an immunostimulatory response against a tumor present at the time of treatment by exposing, a cationic molecule:biologically active molecule complex to a mammalian cell or a foreign tumor cell.

The immune response stimulated by the cationic molecule:biologically active molecule complex may be an apoptotic response, anti-angiogenic response, inflammatory response, humoral response, cellular response, Th1 or Th2 type response, any other immune response sub-classified as an inflammatory response, or any other immunostimulatory response or anti-cancer response known in the art. Additionally, any other immune response known to be generated by CpG motifs, or bacterially or synthetically derived plasmids, is within the practice of the invention. In a preferred embodiment, the immune response is a protective immune response that may provide long term protective immune memory.

The method of the invention preferably comprises a cationic lipid:biologically active molecule complex. While an inflammatory and/or immune response has been observed following the individual administration of both a cationic lipid and an immunologically active nucleic acid sequence, the preferred response of the invention is obtained by the administration of a composition comprising both a cationic lipid and a biologically active molecule.

The invention provides for the use of any cationic lipid compounds. The traditional use of cationic lipids as carriers of biologically active molecules is to facilitate transfection of the biologically active molecule into a cell. Gene therapy requires successful transfection of target cells in a host. Transfection, which is practically useful per se, may generally be defined as a process of introducing an expressible polynucleotide (for example, a gene, a cDNA, or an mRNA) or other biologically active molecule into a cell. Successful expression of the encoding polynucleotide thus transfected leads to production in the cells of a protein. The present invention does not require the transfection of the biologically active molecule or the expression of a transgene. While transfection or expression may be helpful and desired in some situations, stimulation and/or modulation of the inflammatory response or generation of an immune or anti-cancer response may only require delivery of the cationic lipid:biologically active molecule complex to a cell.

Cationic molecules have polar groups that are capable of being positively charged at or around physiological pH. This property is understood in the art to be important in defining how the cationic lipids interact with the many types of biologically active molecules including, for example negatively charged polynucleotides such as DNA. In a preferred embodiment, the invention provides for the use of any cationic lipid and compositions containing them that are useful to facilitate the transport of a biologically active molecule to a cell, tissue, organ, the vascular system, or a body cavity. A number of preferred cationic lipids according to the practice of the invention can be found in U.S. Pat. Nos. 5,747,471 & 5,650,096 and PCT publication WO 98/02191. In addition to cationic lipid compounds, these patents disclose numerous preferred co-lipids, biologically active molecules, formulations, procedures, routes of administration, and dosages. Representative cationic lipids that are useful in the practice of the invention are: 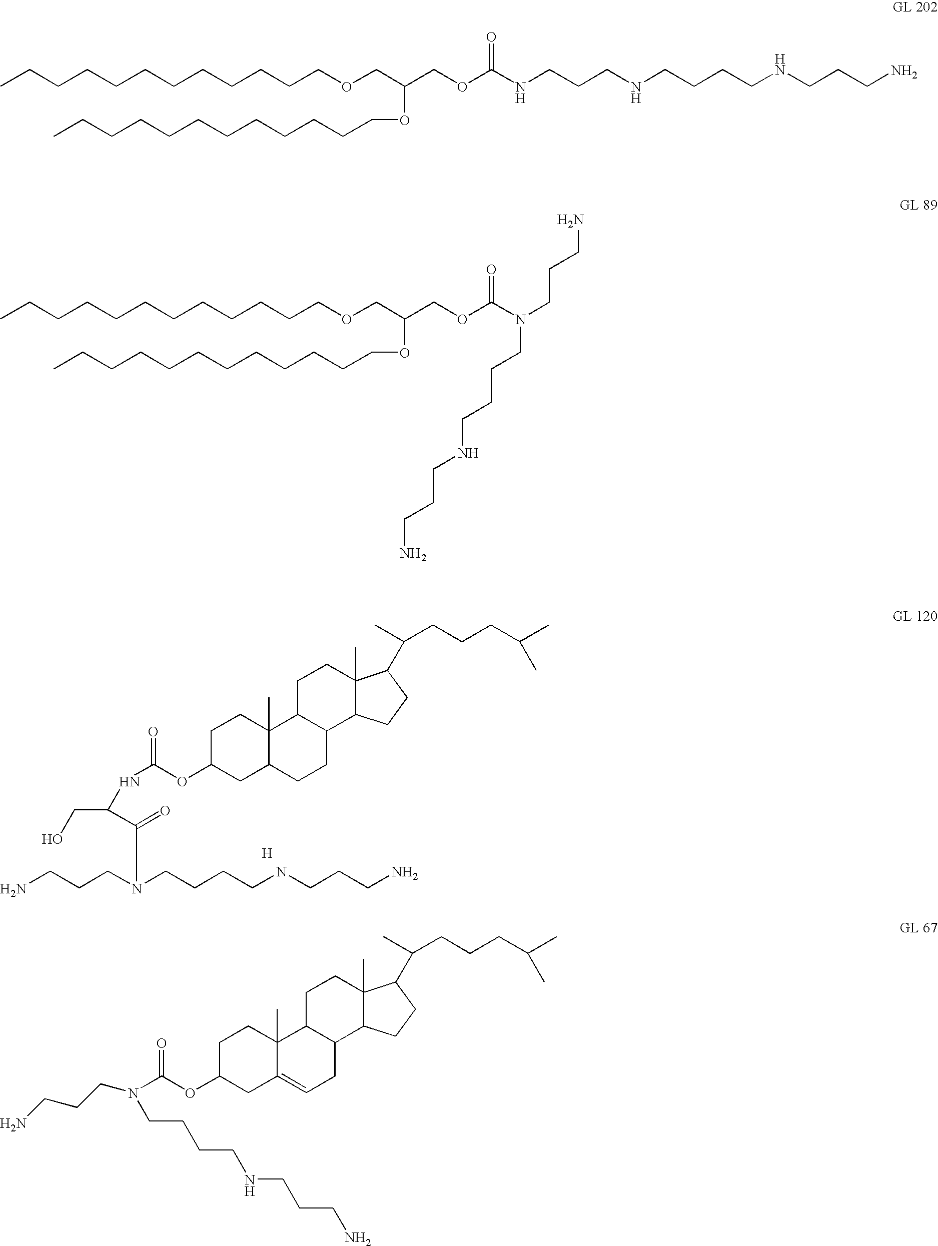
and other lipids that are known in the art including those described in U.S. Pat. Nos. 5,747,471 & 5,650,096 and PCT publication WO 98/02191.

The biologically active molecule is preferable an immunologically active nucleic acid sequence, which may be a plasmid, with a non-expressible or expressible DNA insert. However, biologically active molecules included in the practice of the invention include any representative biologically active molecule that can be delivered to a cell in order to stimulate an inflammatory and/or immune response using the methods of the invention including: oligonucleotides containing bacterial sequences; polynucleotides such as genomic DNA, cDNA, and mRNA; ribosomal RNA; antisense polynucleotides; ribozymes; null vectors or vectors without an expressible insert; and low molecular weight biologically active molecules such as hormones and antibiotics.

The immunologically active nucleic acid sequence may be bacterially, synthetically, or vertebrate derived. However, for most applications, a bacterially or synthetically derived sequence is preferred and more preferably a sequence that contains CpG motifs or more even preferably a high frequency of CpG motifs. CpG motifs of bacterial and synthetic origin which are thought to activate certain immune cells including B cells, monocytes, dendritic cells, macrophages, and natural killer cells are within the practice of the invention. Additionally, CpG motifs which can be used to activate protective immune responses against infection, enhance vaccines, and activate the immune system against cancer cells are within the scope of the invention.

In another subject of the invention, a biologically active molecule with CpG motifs stimulates an immune response or an anti-tumor response against a tumor present at the time of treatment when the cationic molecule:biologically active molecule complex is delivered to a host cell. The invention also provides for a method of stimulating an inflammatory and/or immune response by delivering a immunologically active nucleic acid sequence with CpG motifs using a cationic lipid.

Within the practice the invention, an anti-tumor effect may be generated by exposing a tumor cell to a cationic lipid:biologically active molecule complex. The anti-tumor cell response may preferably be a Th1-type response, a Th2-type response, an inflammatory response, an anti-angiogenic response, a pro-apoptotic response, or any other anti-cancer response known in the art. In a preferred embodiment, a cationic lipid:biologically active molecule complex stimulates a long term adaptive immune response against a tumor cell. The invention also provides for direct administration of the cationic molecule:biologically active molecule complex to a tumor cell in order to generate an a long term adaptive immunostimulatory response and which suppresses or inhibits growth of the tumor cell including administration into the intra-peritoneal, pleural cavity, blood compartment or any other body compartment. Administration may be by injection, intravenously, instillation, inhalation or any other method of administration deemed appropriate by one of sufficient skill in the art including a systemic administration through the vasculature.

Another subject of the invention provides for methods of stimulating an immune response in a mammal by targeting the tumor cell by incorporating targeting agents or using a cationic molecule which targets the cells, tissues, organs, or vasculature in the area of the tumor cell.

The immune response or anti-tumor effect generated by the methods of the invention may be a localized effect, or in a preferred embodiment, the specific immune response may be a systemic response. More preferably, the specific localized or systemic immune response that is generated may be determined by the type of tumor cell that is exposed to the cationic molecule:biologically active molecule complex and/or the type biologically active molecule or cationic molecule exposed to the tumor cell.

Within the practice of the invention, the biologically active molecule may be immunologically active nucleic acid sequence that mayor may not contain an expressible cDNA insert. The methods of invention therefore do not require the expression of a transgene. The subject of the invention also includes the use of an expressible biologically active molecule in the composition or administered as part of a composition in order to generate an immune, inflammatory, or therapeutic response. In the practice of the invention, the methods and compositions of the invention may provide additional therapeutic benefits through the transfection and expression of a biologically active molecule.

Also within the practice of the invention is the administration of compositions comprising a cationic molecule:biologically active molecule complex for the purpose of modulating an inflammatory response. The modulation may be in response to the delivery of the cationic molecule:biologically active molecule complex or the expression of the biologically active molecule and/or the modulation may be regulated by the complex or a transfected biologically active molecule, for example, by using a segment of a plasmid.

Other Lipids Including Co-Lipids

It has been determined that the stability, delivery and transfection-enhancing capability of cationic molecule compositions can be substantially improved by adding to such formulations small additional amounts of one or more derivatized polyethylene glycol compounds. Such enhanced performance is particularly apparent when measured by stability of cationic lipid formulations to storage and manipulation, including in liquid (suspended) form, and when measured by stability during aerosol delivery of such formulations containing a biologically active molecule, particularly polynucleotides.

According to the practice of the invention, any derivative of polyethylene glycol may be part of a cationic molecule formulation. Complexes have been prepared using a variety of PEG derivatives and all of the PEG derivatives, at a certain minimum cationic lipid:PEG derivative ratio have been able to form stable homogeneous complexes. Although the inventors are not limited as to theory, PEG derivatives can stabilize cationic lipid formulations and enhance the delivery and transfecting properties and the affinity of formulations to biologically active molecules. The use of PEG and PEG derivatives enables one to use a higher ratio of biologically active molecules, especially DNA, to lipid. The following references, specifically incorporated by reference herein, contain more information regarding use of PEG derivatives: Simon J. Eastman et al., Human Gene Therapy 8: 765-773 (1997); and Simon J. Eastman et al., Human Gene Therapy 8: 313-322 (1997). Derivatives of polyethylene glycol useful in the practice of the invention include any PEG polymer derivative with a hydrophobic group attached to the PEG polymer.

For pharmaceutical use, the cationic molecule:biologically molecule complexes of the invention may be formulated with one or more additional cationic lipids including those known in the art, or with neutral co-lipids such as dioleoylphosphatidyl-ethanolamine (“DOPE”), to facilitate delivery of the complexes to cells of a host. The use of neutral colipids is optional. Depending on the formulation, including neutral co-lipids may substantially enhance delivery and/or transfection capabilities. Representative neutral co lipids include dioleoylphosphatidylethanolamine (“DOPE”), diphytanoylphosphatidylethanolamine, lyso-phosphatidylethanolamines, other phosphatidyl-ethanolamines, phosphatidylcholines, lyso-phosphatidylcholines, and cholesterol. Use of diphytanoylphosphatidylethanolamine is highly preferred according to the practice of the present invention, as is use of “DOPE”.

Other Carriers & Delivery Vehicles

The invention also provides for a composition that comprises one or more lipids or other carriers that have been employed in the art to effectuate delivery of biologically active molecules to the cells of mammals, and one or more biologically active molecule, wherein said compositions facilitate delivery of effective amounts of the biologically active molecules or lipid complexes. Numerous methods and delivery vehicles are within the practice of the invention including viral vectors; DNA encapsulated in liposomes, lipid delivery vehicles, and naked DNA have been employed to effectuate the delivery of DNA to the cells of mammals. To date, delivery of DNA in vitro, ex vivo, and in vivo has been demonstrated using many of the aforementioned methods.

Other carriers or delivery vehicles that may be included in the compositions of the present invention include viral vectors, adenoviruses, retroviruses, and also non-viral and non-proteinaceous vectors or other alternative approaches that are known in the art to facilitate delivery of biologically active molecules The person skilled in the art will, of course, take care to choose additional carriers or delivery vehicles and/or their concentration in such a way that the desired properties or activity of the invention are not, or are not substantially, impaired by the envisaged addition.

Preparation of Compositions and Administration Thereof

The pharmaceutical compositions of the invention may be formulated to contain one or more additional physiologically acceptable substances that stabilize the compositions for storage, target specific tissues, cells, membranes or organs and/or contribute to the successful intracellular delivery of the cationic lipid:biologically active molecule complex.

The present invention provides for pharmaceutical compositions that facilitate delivery of therapeutically effective amounts of cationic molecule:biologically active molecule complexes. A pharmaceutical composition may comprise a cationic molecule:biologically active molecule complex, lipid or non-lipid carriers, other biologically active molecules, or any other known additives which facilitate delivery of a cationic molecule:biologically active molecule complex.

Pharmaceutical compositions of the invention may facilitate delivery of a cationic molecule:biologically active molecule complexes to numerous cells, tissues and organs such as the gastric mucosa, heart, lung, and solid tumors; cavities and body compartments such as the peritoneal cavity, pleural cavity, blood compartment; and the vascular system and blood cells. Additionally, compositions of the invention facilitate delivery of cationic molecule:biologically active molecule complexes to cells that are maintained in vitro, such as in tissue culture.

Cationic lipid species, PEG derivatives, co-lipids and other carriers and delivery vehicles of the invention may be blended so that two or more species of cationic lipid or PEG derivative, co-lipid or carrier are used, in combination, to facilitate delivery of a cationic lipid:biologically active molecule complex into target cells and/or into subcellular compartments thereof. Cationic lipids of the invention can also be blended for such use with lipids that are known in the art. Additionally, a targeting agent may be coupled to any combination of cationic lipid, PEG derivative, and co-lipid or other lipid or non-lipid formulation that effectuates delivery of a cationic lipid:biologically active molecule complex to a mammalian cell.

The cationic molecule:biologically active molecule complexes may also be used as an adjuvant that can be combined with another drug or treatment to increase or aid its efficacy. For example, a cationic molecule:biologically active molecule complex may be administered with a known tumor antigen including but not limited to proteins, peptides or cDNA. The cationic molecule:biologically active molecule complexes may also be administered with a tumor cell, or tumor cell lysate, etc, that would contain all tumor antigens. This could be either an autologous (from the patient being treated) tumor cell or an allogeneic (from the same tumor type) tumor cell.

Dosages of the pharmaceutical compositions of the invention will vary, depending on factors such as half-life of the biologically-active molecule and the a cationic molecule:biologically active molecule complex, potency of the biologically-active molecule and the a cationic molecule:biologically active molecule complex, half-life of other delivery vehicles, any potential adverse effects of the cationic molecule:biologically active molecule complex or delivery vehicle if present or of degradation products thereof, the route of administration, the condition of the patient, and the like. Such factors are capable of determination by those skilled in the art.

A variety of methods of administration may be used to provide highly accurate dosages of the compositions of the invention. Such preparations can be administered intravenously, orally, parenterally, topically, transmucosally, or by injection of a preparation into a body cavity of the patient, or by using a sustained-release formulation containing a biodegradable material, or by onsite delivery using additional micelles, gels and liposomes.

Nebulizing devices, powder inhalers, dry powder formulations, aerosolized solutions, or other representative of methods that may be used to administer such preparations. The invention provides for a method of administering the complexes by any methods that have been employed in the art to effectuate delivery of biologically active molecules to the cells of mammals.

Additionally, the compositions, which include therapeutic and pharmaceutically acceptable compositions of the invention, can in general be formulated with excipients (such as the carbohydrates lactose, trehalose, sucrose, mannitol, maltose or galactose, and inorganic or organic salts) and may also be lyophilized (and then rehydrated) in the presence of such excipients prior to use. The complexes may be an active ingredient in a pharmaceutical composition that includes carriers, fillers, extenders, dispersants, creams, gels, solutions and other excipients that are common in the pharmaceutical formulatory arts. Conditions of optimized formulation for each Complex of the invention are capable of determination by those skilled in the pharmaceutical art. Selection of optimum concentrations of particular excipients for particular formulations is subject to experimentation, but can be determined by those skilled in the art for each such formulation.

The invention will be further clarified by the following examples, which are intended to be illustrative of the invention, but not limiting thereof.

### EXAMPLES

The following Examples are representative of the practice of the invention.

### Example 1

### Construction and Purification of Plasmid DNA

The construction and characterization of the plasmid vector pCF1-CAT encoding the reporter gene product chloramphenicol acetyltransferase (CAT) has been described previously. See Yew et al. Hum. Gene Ther. 8: 575-584 (1997). pCF1-CAT contains the strong promoter from the human cytomegalovirus immediate-early gene (CMV), an intron, the bovine growth hormone polyadenylation signal sequence, a pUC origin, and the aminoglycoside 3′-phosphotransferase gene that confers resistance to kanamycin. pCF1-null is analogous to pCF1-CAT except that the cDNA for CAT was deleted. pCFA-299-CAT was constructed by digesting pCFA-CAT (identical to pCF1-CAT except for the addition of a small poly linker 5′ of CMV) with Pme I (in the poly linker) and BgI I (in CMV), blunting the ends with the Klenow fragment of DNA polymerase 1, then replicating. This results in deletion of nucleotides −522 to −300 of the CMV promoter.

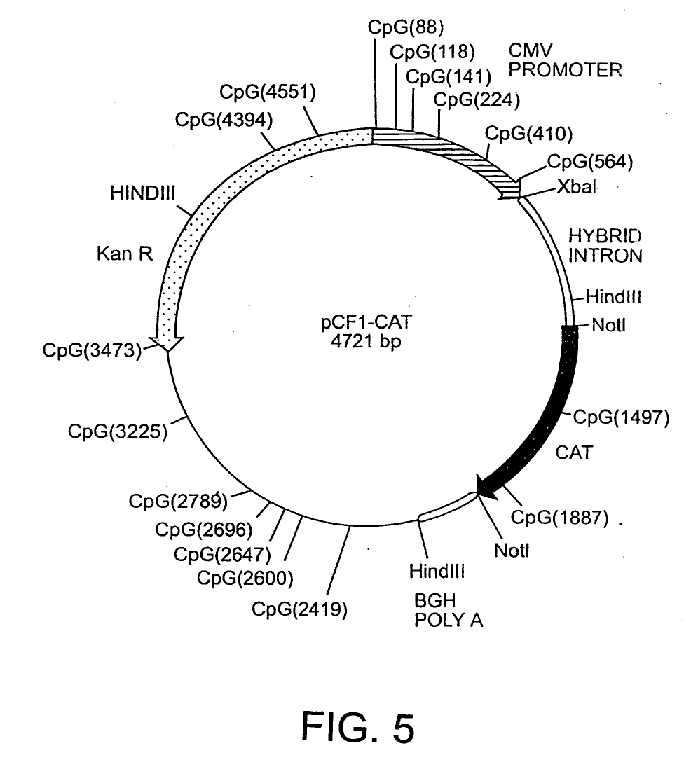

Site-directed mutagenesis was performed using the QuickChange Site-Directed Mutagenesis kit (Stratagene) following the protocol described by the manufacturer. One modification was that multiple sets of oligonucleotides were used simultaneously, allowing mutagenesis of three or more sites in a single reaction. The mutations were confirmed by extensive DNA sequencing and restriction enzyme mapping to check for plasmid integrity. pCFA-299-10M-CAT is deleted of the CpG motifs at nucleotides 88, 118, 141, and 224 (number refers to the C residue within the CpG dinucleotide except where indicated and is based on the pCF1-CAT sequence; see FIG. 5), and contains 10 point mutations at nucleotides 410, 564, 1497 (G to A), 1887, 2419, 2600, 2696, 3473, 4394 (G to A), and 4551.

Plasmid DNA was prepared by bacterial fermentation and purified by ultrafiltration and sequential column chromatography essentially as described previously. See Lee et al., Hum. Gen Ther. 7: 1701-1717 (1996); Scheule et al., Hum. Gene Ther. 8: 689-707 (1997). The purified preparations contained less than 5 endotoxin units/mg of pDNA as determined by a chromogenic LAL assay (BioWhittaker), less than 10 μg protein/mg pDNA as determined by the micro BCA assay (Pierce), and less than 10 μg of bacterial chromosomal DNA/mg of pDNA as determined by a dot-blot assay. They were also essentially free of detectable RNA and exhibited spectrophotometric A_{260/280} ratios of between 1.8 and 2.0.

### Example 2

### In Vitro Methylation of pDNA

Plasmid DNAs were methylated in vitro in a 5 ml reaction containing 1×NEB buffer 2 [50 mM NaCl, 10 mM Tds-HCI, pH 7.9, 10 mM MgCI2, 1 mM dithiothreitol], 160 μM S-adenosylmethionine (SAM), 1-3 mg of pDNA, and 1 U of Sss I methylase (New England Biolabs) per μg of pDNA. The mixture was incubated at 37° C. for 18 h. Additional SAM was added to a concentration of ISO μM after 4 h of incubation. Mock treatment of pDNA used the same procedure except the Sss I methylase was omitted. Methylated and mock-treated pDNA was centrifuged through a Millipore Probind column, ethanol precipitated, and washed with 70% (v/v) ethanol. The pDNA was resuspended in water to a final concentration of approximately 3 mg/ml. In experiments to examine the effects of Sss I-mediated methylation of pDNA, mock-methylated pDNA was always used as a control.

The extent of pDNA methylation was assessed by digesting 0.2-0.5 μg of the treated pDNA with 10 U BstU I or Hpa II for 1 h, then analyzing the pDNA by agarose gal electrophoresis. Methylated pDNA was protected from BstU I and Hpa II digestion whereas unmethylated or partially methylated pDNA was cleaved. Gel analysis showed that the methylated pDNA was completely protected from either BstU I or Hpa II digestion.

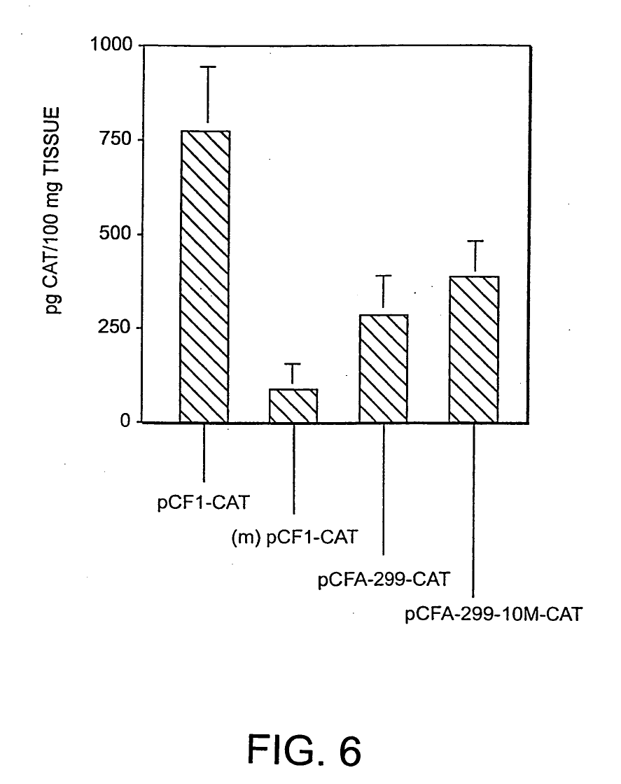

The plasmids used in these studies were highly purified and contained predominantly the supercoiled form, less than 1 endotoxin unit/mg of plasmid and were free of infectious contaminants as determined using a bioburden assay. To assess the role of methylation of CpG dinucleotides in the plasmid DNA on lung inflammation, the purified pDNAs were either methylated or mock methylated in vitro using E. coli Sss I methylase. This enzyme methylates the cytosine residue (C5) within all CG dinucleotides. The extent of methylation was assessed by monitoring the susceptibility of the modified plasmids to digestion by BstU I or Hpa II but not Msp I. An Sss 1-methylated but not the mock-methylated plasmids were completely protected from digestion with BstU I and Hpa II. Methylation of pCF1-CAT also resulted in an approximately 5 fold reduction in expression levels following intranasal administration into lungs of BALB/c mice (FIG. 6).

Cytokine levels in the mouse BALF were quantitated using enzyme-linked immunosorbent assay (ELISA) kits as specified by the manufacturers. IFN-γ, TNF-α, IL1-α, IL-1β, IL-10 and IL-6 ELISA kits were from Genzyme Corporation, while mKC, MIP-2 and GM-CSF ELISA kits were from R&D Systems, and the Leukotriene B4 ELISA kit was from Perseptive Diagnostics.

The procedures for processing the lung tissues and assay of CAT enzymatic activity have been described elsewhere. See Lee et al., Hum. Gene Ther. 7: 1701-1717 (1996); Yew et al., Hum. Gene Ther. 8: 575-84 (1997).

### Example 3

### Nasal Instillation of Cationic Lipid:pDNA Complexes into Mice

The cationic lipid:pDNA complexes were formed by mixing equal volumes of GL67:DOPE (1:2) with pDNA as described previously (Lee et al., Hum. Gene Ther. 7: 1701-1717, (1996)) to a final concentration of 0.6:1.2:3.6 mM (GL-67:DOPE:pDNA) or 0.3:0.6:1.8 mM, as indicated in the figure legends. The DNA concentration is expressed in terms of nucleotides, using an average nucleotide molecular weight of 330 daltons. BALB/c mice were instilled intranasally with 100 μl of complex as described. See Scheule et al., Hum. Gene Ther. 8: 689-707 (1997). The animals were euthanized and their lungs were lavaged 24 h post-instillation using phosphate-buffered saline (PBS). The recovered BALF were centrifuged at 1,500 rpm for 4 min, and the resulting supernatants were removed and frozen at −80° C. for subsequent cytokine analysis. The cell pellets were resuspended in PBS for microscopic determination of cell number and cell types.

### Example 4

### Composition of Bronchoalveolar Lavage Fluid After Administration of Cationic Lipid:pDNA Complexes Harboring Either Methylated or Unmethylated pDNA

The Sss 1-methylated (m)pDNA or unmethylated pDNA were complexed with the cationic lipid GL-67 and then instilled intranasally into BALB/c mice. Separate groups of mice were instilled with either (m)pDNA or unmethylated pDNA alone, or vehicle, and their bronchoalveolar lavage fluids collected for analysis at 24 h post-treatment.

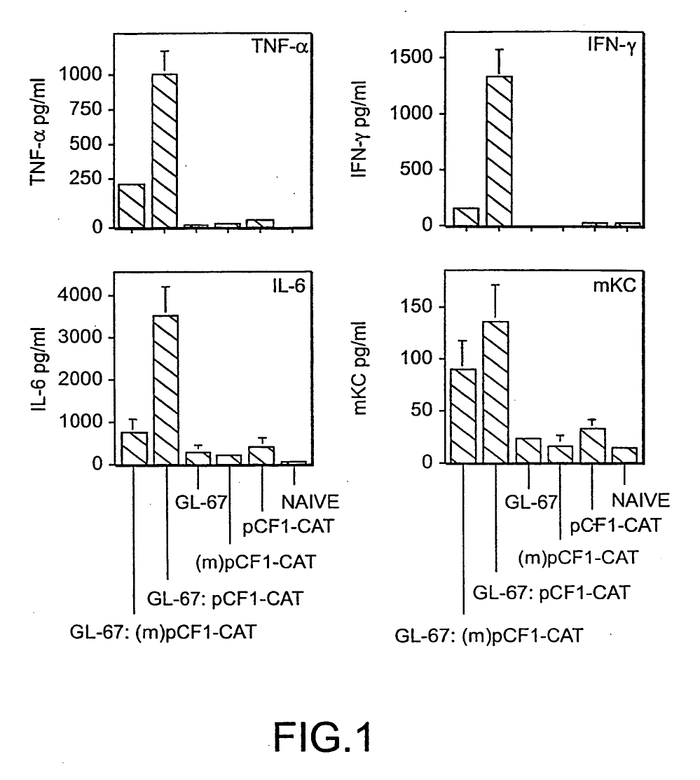

To determine whether methylation of pDNA affected the inflammatory response in the lungs, we measured the levels of several different cytokines in the BALF 24 h after instillation. Significantly higher levels of TNF-α, IFN-γ, and to a lesser extent IL-6, were found in the BALF of mice that received GL-67:pCF1-CAT when compared to those administered GL-67:(m)pCF1-CAT (FIG. 1). Levels of murine KC were also elevated following instillation of the cationic lipid:pDNA complexes but there was no significant difference in the levels of the cytokine induced by either methylated or unmethylated pDNA complexed with GL-67. In contrast, low levels of these four cytokine were present after instillation with GL-67 alone, (m)pCF1-CAT alone or unmethylated pCF1-CAT alone (FIG. 1). However, although the levels of TNF-α, IFN-γ and IL-6 were low in the BALF of animals treated with free pDNA compared to complexed pDNA, the levels of these cytokines were invariably higher in the group that received free unmethylated pDNA alone than in the group administered (m)pCF1-CAT. The cytokines IL-10, leukotriene B-4, IL-1β, IL-1α, MIP-2, and GM-CSF were also assayed but in each case the levels were low and indistinguishable from those attained in naive animals. These results indicated that unmethylated pDNA was inflammatory in the lung and that this response was exacerbated when the pDNA was present in a complex with GL-67. Furthermore, of the cytokines induced by administration of GL-67:pCF1-CAT complexes to the lung, TNF-α, IFN-γ and a proportion of the IL-6 were primarily due to the presence of unmethylated pDNA. The cationic lipid GL-67 did not contribute significantly to the cytokine induction in the BALF with the exception of KC where it appeared to work in concert with pDNA to increase its level.

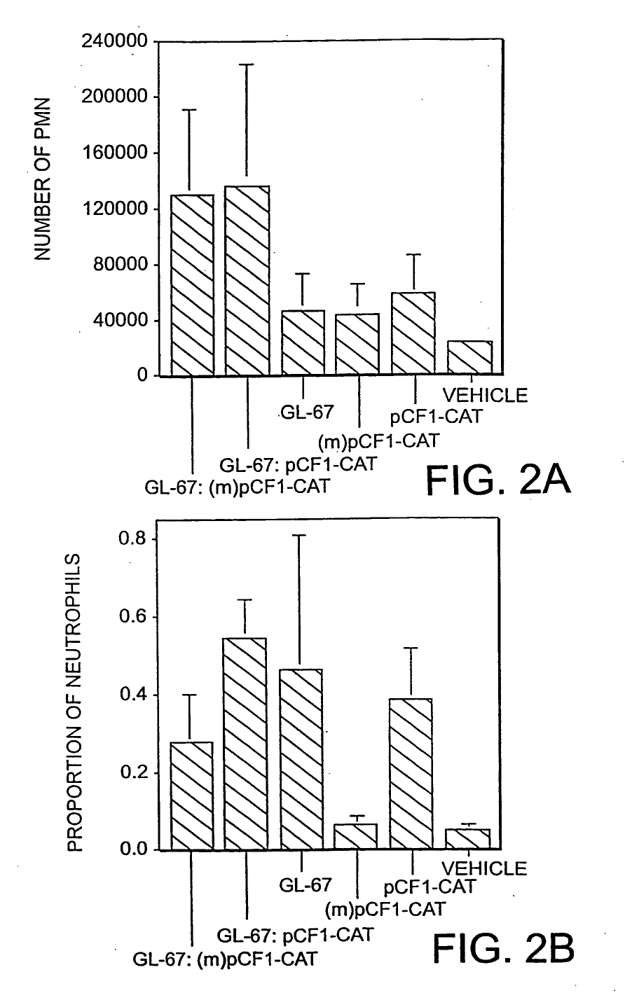

The character of the inflammatory response induced by GL-67:pCF1-CAT was also evaluated by measuring the total number of cells and the differential counts recovered in the BALF of the treated animals. Elevated numbers of polymorphonuclear (PMN) leukocytes were present in the BALF of mice that were instilled with GL-67:pDNA compared to mice that received either GL-67 alone or pDNA alone (FIG. 2A). The methylation status of the pDNA in the GL-67:pDNA complex did not significantly affect the overall cell number. However, animals administered (m)pCF1-CAT alone (4 separate experiments) consistently showed a slight reduction in the total number of PMN leukocytes in comparison to those that received pCF1-CAT. An analysis of the different cell types showed an increased proportion of neutrophils in mice that received GL-67:pCF1-CAT compared to mice that received GL-67:(m)pCF1-CAT (FIG. 2B). This increase was also observed after instillation of pCF1-CAT alone compared to (m)pCF1-CAT alone. Together, these data indicate that the induction in cellular influx was mediated by both the cationic lipid and pDNA. However, administration of unmethylated pDNA rather than methylated pDNA into the lung can result in an increase in the number of PMN leukocytes, particularly neutrophils, in the BALF.

Since pCF1-CAT expresses high levels of the CAT reporter enzyme, which is a bacterial protein, there was the possibility that the cytokine response was due to the expression of the foreign protein. Therefore, experiments were repeated using a plasmid vector that contained the same plasmid backbone but lacked any transgene (pCF1-null). The cytokine induction profile after administration of methylated or unmethylated pCF1-null complexed with GL-67 was essentially identical to that attained with pCF1-CAT. This confirmed that the plasmid DNA itself, and not expression of the bacterial CAT, was responsible for the observed cytokine induction.

### Example 5

### Dose-Dependent Relationship Between Unmethylated pDNA and Cytokine Levels

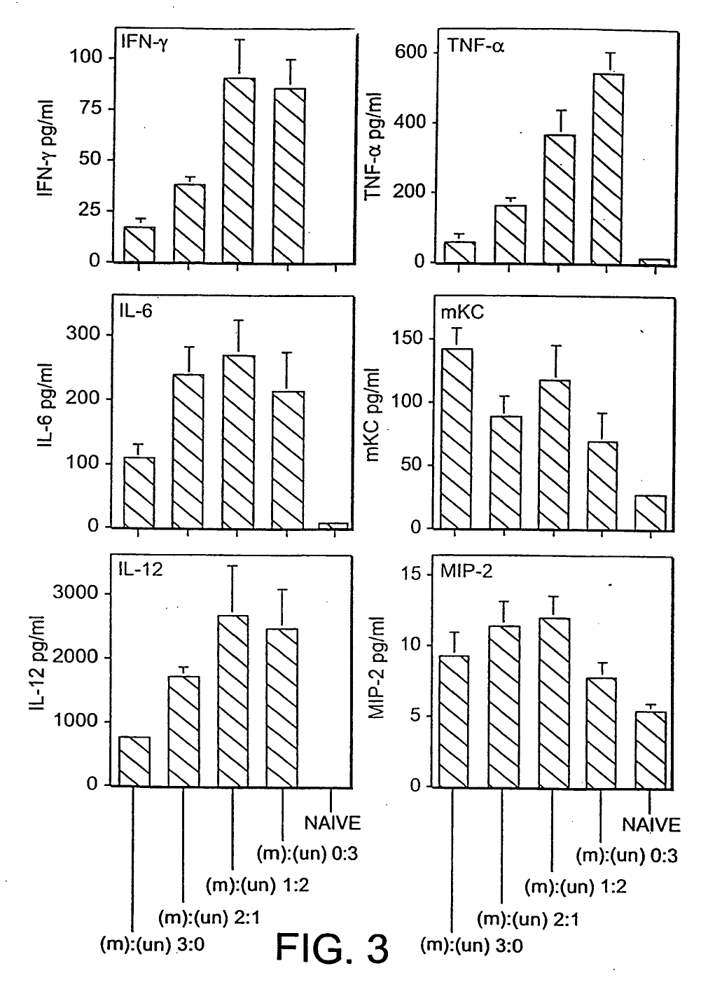

To determine whether there was a dose-dependent relationship between the amount of unmethylated pDNA administered to the lung and the levels of induced cytokines, (m)pCF1-CAT was mixed with pCF1-CAT at different ratios before complexing with GL 67. The dose of GL-67 and the total amount of nucleotides delivered remained constant. In this experiment MIP-2 and IL-12 were assayed in addition to TNF-α, IFN-γ, IL-6, and mKC. As the proportion of unmethylated pCF1-CAT in the complex increased, there was a corresponding increase in the levels of TNF-α, IFN-γ, IL-6, and IL-12 (FIG. 3). With IFN-γ, IL-6 and IL-12, the stimulated increase in cytokine levels was maximal when the ratio of methylated:unmethylated pDNA was 1:2. This dose-dependent relationship supports the proposal that the induction of TNF-α, IFN-γ, IL-6, and IL-12 in the BALF were in direct response to the presence of unmethylated pDNA. This trend was not observed for either KC or MIP-2, consistent with the observations above (FIG. 3).

### Example 6

### Histopathological Changes in the Lung After Administration of Cationic Lipid:Methylated pDNA Complexes

The histopathological changes within BALB/c mouse lungs following administration of either cationic lipid alone, pDNA alone, or cationic lipid:pDNA complexes were also examined. BALB/c mice were instilled intranasally with GL-67:(m)pCF1-CAT, GL67:pCF1-CAT, GL-67 alone, (m)pCF1-CAT, pCF1-CAT, or water (vehicle control). Mice were sacrificed 2 days post-instillation and the lungs were processed for histological examination in a blinded manner. Histopathology.

Lungs were fixed by inflation at 30 cm of H₂O pressure with 2% paraformaldehyde and 0.2% glutaraldehyde. Representative samples were taken from each lung lobe, embedded in glycol methacrylate, sectioned and stained with hematoxylin and eosin. Histopathology on the lung was evaluated in a blinded fashion and graded subjectively using a scale of 0 to 4, where a score of 0 indicates no abnormal findings and a score of 4 reflects severe changes with intense infiltrates. See Scheule et al., Hum. Gene Ther. 8: 689-707 (1997).

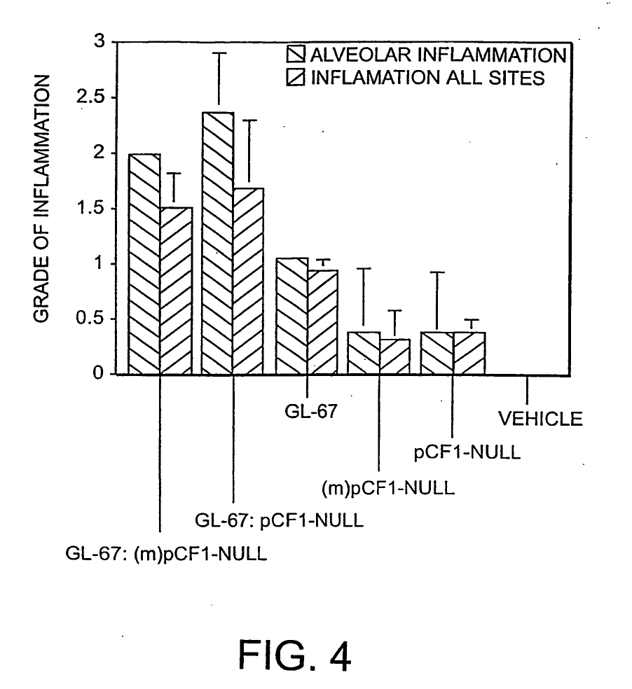

Multifocal areas of alveolar inflammation were observed in mice that received GL67:pDNA complexes. The extent of lung inflammation was graded using a scale from 0 to 4, with 0 indicating no abnormalities, 1 indicating a minimal change, 2 a mild change, 3 a moderate change, and 4 representing severe changes from a normal lung (FIG. 4). There was no significant difference in the inflammation score of lungs that received GL-67:pDNA compared to lungs that received GL-67:(m)pDNA complex. Lungs that received GL-67 alone were scored slightly lower than lungs that received lipid:pDNA complex, while minimal inflammation was observed in lungs that received either pDNA or (m)pDNA alone.

These results indicated that the presence of unmethylated CpG motifs on the pDNA did not grossly affect the histopathological changes observed in the lung after administration of cationic lipid:pDNA complexes. Furthermore, the majority of the histological changes observed upon administration of the complexes was mediated by the cationic lipid component.

### Example 7

### Effect of Mutating Immunostimulatory CpG Motifs Within pCF1-CAT

Since a subset of the unmethylated CpG dinucleotides present in pCF1-CAT appears responsible for the majority of the cytokine response, then elimination of these particular CpG motifs should reduce the level of induction. There are 17 motifs in pCF1-CAT having the sequence 5′-RRCGYY-3, which have been previously shown to be the sequence context in which the CpG motif was found to be most immunostimulatory (FIG. 5). Fourteen of these motifs were eliminated by either deletion or site-directed mutagenesis. The four CpG motifs located within the CMV promoter (at nucleotide positions 88, 118, 141 and 224) were removed by deletion of a 400 bp fragment containing a portion of the upstream enhancer region, to create pCFA-299-CAT (FIG. 5). Ten of the thirteen remaining motifs (at positions 410, 564, 1497, 1887, 2419, 2600, 2696, 3473, 4394 and 4551) were modified using site-directed mutagenesis to create pCFA-299-10M-CAT (FIG. 5). The cytosine residue in each motif was mutated to a thymidine residue in each case, with the exception of one motif (nucleotide 1497) within the coding sequence for CAT, and one motif (nucleotide 4394) within the kanamycin resistance gene. With these two motifs, in order to preserve the coding sequence for the respective proteins, the guanidine residue of the CpG dinucleotide was changed to an adenosine residue.

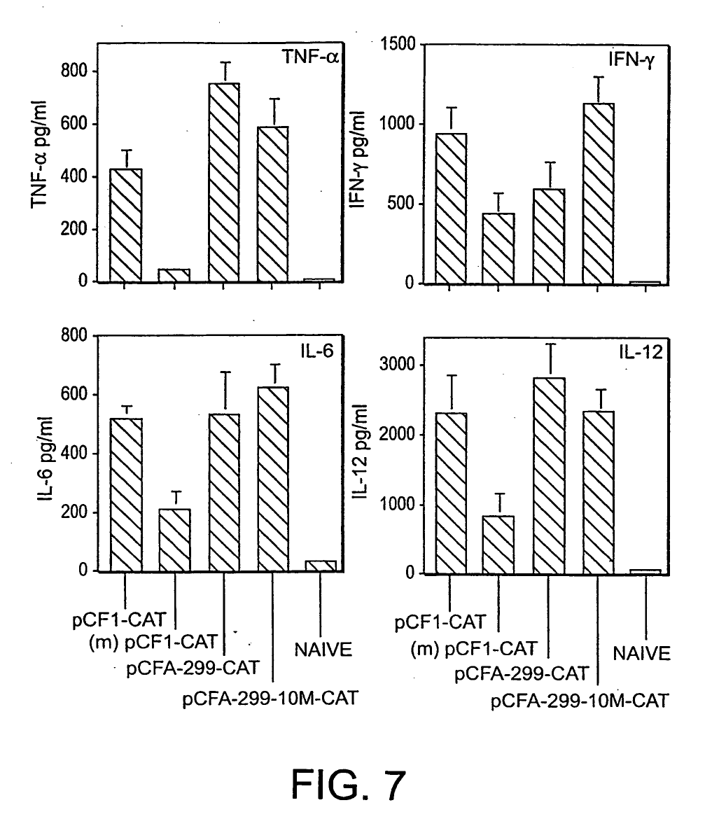

The plasmids, pCF1-CAT, (m)pCF1-CAT, pCFA-299-CAT, and pCFA-299-10MCAT were complexed with cationic lipid GL-67 then instilled intranasally into BALB/c mice. Twenty-four hours after instillation, BALF was collected for cytokine analysis and the lungs harvested for CAT assays. Expression from pCFA-299-CAT, containing the truncated CMV promoter, was approximately one-third that of pCF1-CAT (FIG. 6). The expression from pCFA-299-10M-CAT was equivalent to pCFA-299-CA T, indicating that the introduction of the 10 point mutations did not affect transgene expression (FIG. 6). As before, high levels of TNF-α, IFN-γ, IL-6, and IL-12 were present in the BALF of mice that received unmethylated pCF1-CAT (FIG. 7). However, equally high levels of these cytokines were also observed with pCFA-299-CAT and pCFA-299-10M-CAT. Therefore, reducing the content of CpG motifs within the plasmid did not reduce its ability to elevate cytokine levels in the lung. This suggests that other immunostimulatory motifs in addition those harboring the consensus 5′-RRCGYY-3′ are necessary to stimulate the desired inflammatory response.

### Example 8

### Effect of Cationic Lipid:Biologically Active Molecule Complexes on Tumor Growth B16 Melanoma Subcutaneous Model

B16/FO cells (5×10⁴) were implanted subcutaneously in C57/BL6 mice (8/group) and allowed to grow for 12 days until they were 3-4 mm in anyone dimension. Tumors were injected with lipid:pDNA complexes bearing either the purine nucleoside phosphorylase (PNP) gene, which catalyzes the conversion of several non-toxic deoyadenosine analogs to highly toxic adenine analogs, or the b-gal gene (control) on days 1 and 3. Animals were administered prodrug (Fludara) intraperitoneally on days 2-7. Compared to untreated animals, the growth of tumors on animals treated with complex, regardless of the transgene, were inhibited by ˜60%. In other words, inhibition of tumor growth was achieved even with the intratumoral injection of a control transgene.

B16 Melanoma Lung Metastasis Model-Lung Mets

On day 0, 1×10⁵ B16/F1O cells were injected intravenously in C57/BL6 mice. On days 5 and 10, mice were treated with an intravenous injection (100 μl) of GL67:pCFA-null complexes. On day 14 mice were sacrificed, lungs excised, fixed and placed in Fekete's solution. The number of lung metastases were counted. Untreated animals had 26±4 (mean±SEM) mets, while the group treated with GL67:pCFA-null had 9.5±2.5 mets, indicating significant (p=0.017) efficacy of a lipid:pDNA complex in the absence of an expressing transgene in this model.

B16 Melanoma Lung Metastasis Model—Survival

On day 0, B16/F10 cells were injected intravenously in C57/BL6 mice. On days 5, 10, 15 and 18, one group of mice was treated with an intravenous injection of GL67:pCFA-null complexes. All mice were followed for survival. The untreated group had a median survival of 27±1.5 days, while the group treated with GL67:pCFA-null complexes exhibited a median survival of 34±1 days, a statistically significant (p=0.0019; Logrank) increase.

B16 Melanoma Lung Metastasis Model—Survival

In a repeat of the above survival experiment, mice were treated intravenously with GL67:pCFA-Null complexes at either 0.5:2 (Low dose) or 2:2 mM (High dose). Treatment resulted in increased median survival for both groups relative to a control, untreated group of animals, which had a median survival of 26.8±0.6 days. The high dose and low dose groups had median survivals of 31.6±1.5 and 34.4±1.2 days, respectively, which were significantly different from control at p values of <0.01 and <0.0001, respectively.

NuTu/Fischer Rat Ovarian Cancer Model

The ovarian epithelial carcinoma cell NuTu19 is syngeneic for the Fischer 344 rat. See Rose, G. S., et al. Am J Obstet Gvnecol 175: 593-599 (1996). On day 0, 1×10⁶ tumor cells in 1 ml were inoculated into the peritoneal cavities of F344 rats. On days 3, 6, and 9, groups (10 animals/group) of animals were treated with 2 ml of either saline or GL67:pCF1bgal (at a 0.5:2 mM molar ratio), a control vector. The group treated with saline had a median survival of 92 days, while the group treated with complex had a median survival of 156 days. These data show a significant effect on survival generated by multiple administrations of a control vector.

MOT Model of Ovarian Cancer

In the mouse ovarian teratoma (MOT) model (Fekete, E. et al., Cancer Res. 12:438443 (1952)), tumor cells were implanted into the peritoneal cavity of C3He/FeJ mice (10 mice/group). On three occasions, the mice were treated with saline or GL67:pNull complexes (in saline) by instillation into the peritoneal cavity. The pNull vector is a pCFA backbone without an expressible cDNA insert.

As shown below, all the saline-treated animals died; there were no long term survivors. However, when tumor-bearing animals were treated with GL67:pNull complexes, the percentage of long-term survivors ranged from zero to 70%, depending on the cationic lipid:DNA ratio. Importantly, when these long-term survivors were rechallenged with MOT tumor cells, the percentage of animals that rejected this challenge also ranged from 0 to 70%. This result indicates a formulation-dependent generation of a protective, memory-based immune response that was systemic in nature.

| | | Complex | | | | | Survival |
|---|---|---|---|---|---|---|---|
| | | | | Ration | | Tumor Free | After |
| | | Lipid | DNA | (lipid:DNA) | Treatment | Survival | Rechallenge |
| Group | Plasmid | (nmol) | (μg) | mM | Days | (%) | (%) |
| 1 | Saline | — | — | — | 1, 8, 15 | 0 | — |
| 2 | pNull | 100 | 16.5 | 1:0.5 | 1, 8, 15 | 50 | 20 |
| 3 | pNull | 100 | 66 | 1:2 | 1, 8, 15 | 30 | 67 |
| 4 | pNull | 100 | 132 | 1:4 | 1, 8, 15 | 30 | 0 |
| 5 | pNull | 100 | 16.5 | 1:0.5 | 2, 9, 16 | 70 | 14 |
| 6 | pNull | 100 | 66 | 1:2 | 2, 9, 16 | 40 | 25 |
| 7 | pNull | 100 | 132 | 1:4 | 2, 9, 16 | 50 | 0 |

### Example 9

### Use of Cationic Lipid:Bacterial Genomic DNA as a Tumor Suppressant

AB12 Mesothelioma Model

AB12 is a murine mesothelioma cell line. BALB/c mice were inoculated untraperitoneally with AB12 mesothelioma cells on day 0. At three time points, days 6, 10 and 14, each group of mice were dosed intraperitoneally with one of the following formulations:
Group A: 50 μg bacterial genomic DNA (cut into ˜4 kb fragments);
Group B: 100 μg bacterial genomic DNA (cut into ˜4 kb fragments);
Group C: 200 μg bacterial genomic DNA (cut into ˜4 kb fragments);
Group D: 100 μg bacterial genomic DNA (cut into ˜4 kb fragments) complexed with cationic lipid GL67 at a 1:4 molar ratio (GL67:DNA); and
Group E: saline.

By 20 days post tumor cell inoculation, there were no surviving mice from the control group, Group E. The results did, however, demonstrate a dose-dependent survival advantage of bacterial genomic DNA. Mice from Group B survived up until day 34 while mice from Group C survived until day 47. At day 60, approximately 12% of the mice from Group C were still alive.

Most surprisingly, there was a significant survival enhancement for the mice treated with the bacterial genomic DNA complexed with cationic lipid GL67. At day 60, post-tumor cell inoculation, 100% of the mice treated with this complex were still alive.

Rat Model of Ovarian Cancer

Administration of bacterial genomic DNA complexed with cationic lipid GL67 also demonstrated efficacy in the OVCA rat model of ovarian cancer. Each group of rats received an intraperitoneal inoculation of tumor cells at day 0. Following the inoculation of tumor cells, each group of rats received an intraperitoneal dose of one of the following formulations at days 6, 10, 14, and 18:
Group A: bacterial genomic DNA (E. coli DNA)
Group B: bacterial genomic DNA (E. coli DNA) complexed with cationic lipid GL67 at a GL67:DNA molar ratio of 1:4.
Group C: saline.

The results demonstrated a significant survival advantage over the control groups for the group of rats treated with GL 67:DNA complex. For example, less than 30% of the rats treated with saline were alive 25 days post-tumor cell inoculation, while approximately 30% of the rats treated with bacterial genomic DNA survived past 26 days. The rats treated with bacterial genomic DNA complexed with a cationic lipid, however, had a survival rate of greater than 70% 45 days post-tumor cell inoculation. This data demonstrates that the therapeutic effect is not limited to mouse tumor models.

M3 Melanoma Model

On day 0, mice were inoculated intraperitoneally with M3 melanoma cells. Following the inoculation of M3 tumor cells, the mice were treated on days 6, 11, 14, and 18 with either the GL67:pNull complex, which is cationic lipid GL67 complexed to a null vector (a vector without an expressible insert), or were left untreated (control). All untreated control animals died by day 40, while greater than 85% of the animals treated with the GL 67:pNull complex were alive on day 68.

On day 68, the surviving animals were rechallenged subcutaneously with M3 tumor cells. A naive group of animals was also challenged with these same cells in parallel. All the animals in the naive group died by day 105, while approximately 40% of the animals that had been originally treated with the GL 67:pNull complex survived not only the initial intraperitoneal tumor cells but also the secondary subcutaneous challenge. These results indicate generation of a protective, memory-based immune response.

The M3 melanoma model was also used to demonstrate that this surprising efficacy cannot be achieved with the components of the lipid:DNA complex, but only with the intact complex. Following the intraperitoneal inoculation on day 0 of M3 tumor cells, groups of mice were treated on days 5, 10, 14, and 18 with either GL 67:pNull complexes (lipid:pNull), an equivalent amount of GL 67 (lipid alone), an equivalent amount of pNull DNA (pNull vector alone), or were untreated.

While GL 67 alone showed some benefit, with about 35% of the mice surviving more than 50 days post tumor cell inoculation, none of the mice treated with the pNull DNA alone or the control survived past 48 days. A significant protection, however, resulted from treatment with the GL 67:pNull complexes, where all of the animals survived at least to day 50. These results not only demonstrate efficacy in an intraperitoneal model of melanoma, they also show that this efficacy cannot be achieved with the individual components of the lipid:DNA complex. This significant efficacy is only observed with the intact complex.

It will be apparent to those skilled in the art that various modifications and variations can be made in the compositions and methods of the present invention without departing from the spirit or scope of the invention. Thus, it is intended that the present description cover the modifications and variations of this invention provided that they come within the scope of the following claims and their equivalents.

1. A method of generating an anti-tumor cell immune response in a tumor-bearing mammal comprising the step of administering to said mammal a composition comprising a complex, said complex comprising: a cationic lipid and an immunologically active nucleic acid sequence without an expressible cDNA insert, wherein said composition is administered in an amount effective to stimulate an immune response against said tumor.

2. A method according to claim 1, wherein said immunologically active nucleic acid sequence is a bacterially derived plasmid.

3. A method according to claim 2, wherein said bacterially derived plasmid comprises CpG rich motifs.

4. A method according to claim, wherein said step of administering is accomplished by intra-tumoral administration or administration into a body cavity compartment containing a tumor.

5. A method according to claim 1, wherein said step of administering is chosen from aerosolization, intravenous injection, intraperitoneal, intranasal, topical, and transmucosal administration.

6. A method according to claim 1, wherein said anti-tumor cell response is a systemic response.

7. A method of generating a protective anti-tumor cell immune response in a tumor-bearing mammal comprising the step of
administering to said mammal a composition comprising a complex, wherein said complex comprises a cationic lipid and an immunologically active nucleic acid sequence that does not encode for an expressible tumor associated antigen, wherein said complex is administered in an amount effective to stimulate an immune response against said tumor.

8. A method according to claim 7, wherein said immunologically active nucleic acid sequence is not capable of transcription or translation of a biologically active peptide in said mammal.

9. A method according to claim 7, wherein said immunologically active nucleic acid sequence is bacterially derived.

10. A method according to claim 7, wherein said immunologically active nucleic acid sequence is a plasmid.

11. A method according to claim 7, wherein said immunologically active nucleic acid sequence comprises genomic bacterial DNA.

12. A method according to claim 7, wherein said immunologically active nucleic acid sequence is a fragment.

13. A method according to claim 7, wherein said immunologically active nucleic acid sequence comprises CpG rich motifs.

14. A method according to claim 7, wherein said step of administering is accomplished by intra-tumoral administration or administration into a body cavity compartment containing a tumor.

15. A method according to claim 7, wherein said step of administering is chosen from aerosolization, intravenous injections, intraperitoneal, intranasal, topical, and transmucosal administration.

16. A method according to claim 7, wherein said protective anti-tumor cell immune response is a systemic response.

17. A method of increasing the efficacy of a tumor antigen comprising the administration of said tumor antigen and an adjuvant, wherein said adjuvant comprises
a cationic molecule:immunologically active nucleic acid sequence complex wherein said immunologically active nucleic acid sequence is without an expressible cDNA insert.

18. A composition for generating a protective anti-tumor cell immune response in a tumor-bearing mammal consisting essentially of:
a cationic lipid; and
a immunologically active nucleic acid sequence without an expressible cDNA insert, and optionally, an adjuvant.

19. A composition according to claim 18 wherein said cationic lipid is GL-67.

20. A method of generating an anti-tumor cell immune response in a mammal comprising the step of administering to said mammal a composition comprising GL-67, and an immunologically active nucleic acid sequence without an expressible cDNA insert, in an amount effective to stimulate said anti-tumor cell immune response.

21. A method according to claim 7, wherein said nucleic acid sequence is selected from the group comprising plasmid DNA, genomic DNA, messenger RNA, and ribosomal RNA.

22. A method according to claim 7, wherein said immune response comprises immune memory against the tumor.

23. A method according to claim 22, wherein said immune response comprises an adaptive immune response against the tumor.

24. A method according to claim 7, wherein said immune response reduces tumor burden distal to the treatment site.

25. A method according to claim 7, wherein said immune response comprises an immune response selected from one of the following responses: an inflammatory response, a humoral response, a cellular response, a Th1 type response, or a Th2 type response.

26. A method according to claim 25, wherein said immune response reduces tumor burden distal to the treatment site.

27. A method according to claim 7, wherein said immune response prolongs the survival of a tumor-bearing mammal.

28. A method according to claim 25, wherein said immune response prolongs the survival of a tumor-bearing mammal.
